(19) United States
(12) Patent Application Publication
(10) Pub. No.: US 2019/0302059 A1
(43) Pub. Date: Oct. 3, 2019

(54) RESONANCE FREQUENCY SHIFT SENSORS

(71) Applicant: Iowa State University Research Foundation, Inc., Ames, IA (US)

(72) Inventors: Nigel Forest Reuel, Ames, IA (US); Sadaf Charkhabi, Ames, IA (US)

(21) Appl. No.: 16/366,287

(22) Filed: Mar. 27, 2019

Related U.S. Application Data
(60) Provisional application No. 62/649,162, filed on Mar. 28, 2018.

(51) Int. Cl. G01N 29/02; H01P 7/00; H01P 11/00; C12Q 1/37

(52) U.S. Cl. CPC G01N 29/022; C12Q 1/37; H01P 11/008; H01P 7/005

(57) ABSTRACT

Various embodiments disclosed relate to a resonator. The resonator can be used for detecting enzymatic activity. The resonator includes an electronically conductive segment. The resonator further includes a polymeric component coating at least a portion of the electronically conductive segment. The resonator further includes a substrate for one or more enzymes. The substrate is disposed on the polymeric component.

### CROSS-REFERENCE TO RELATED APPLICATION

This application claims the benefit of priority to U.S. Provisional Patent Application Ser. No. 62/649,162 entitled “RESONANCE FREQUENCY SHIFT SENSORS,” filed Mar. 28, 2018, the disclosure of which is incorporated herein in its entirety by reference.

### BACKGROUND

Determining the presence and activity of an enzyme can be useful in many different contexts. In some applications, this can be done indirectly by detecting the presence of byproducts of the reaction between an enzyme and substrate. Indirect detection can be unreliable and potentially expensive. It may, therefore, be desirable to develop improved detection methods and assemblies.

### SUMMARY OF THE DISCLOSURE

Various embodiments disclosed relate to a resonator. The resonator can be used for detecting enzymatic activity. The resonator includes an electronically conductive segment. The resonator further includes a polymeric component coating at least a portion of the electronically conductive segment. The resonator further includes a substrate for one or more enzymes. The substrate is disposed on the polymeric component.

Various further embodiments disclosed relate to a method of detecting enzymatic activity. The method includes measuring a first resonance frequency of a resonator. The resonator includes an electronically conductive segment. The resonator further includes a polymeric component coating at least a portion of the electronically conductive segment. The resonator further includes a substrate for one or more enzymes. The substrate is disposed on the polymeric component. The method further includes contacting the substrate of the resonator with an enzyme. The method further includes measuring a second resonance frequency of the resonator following contacting the substrate with the resonator.

Various further embodiments disclosed relate to a method of making a resonator. The resonator includes an electronically conductive segment. The resonator further includes a polymeric component coating at least a portion of the electronically conductive segment. The resonator further includes a substrate for one or more enzymes. The substrate is disposed on the polymeric component. The method includes providing or receiving the electronically conductive segment at least partially coated with the polymeric component. The method further includes etching the electronically conductive segment to form a pattern therein. The method further includes contacting the polymeric component with the substrate.

There are various advantages to using the systems and methods of the instant disclosure, some of which are unexpected. For example, according to some embodiments, inexpensive, flexible, wireless, resonant sensors can be rapidly fabricated using a copper-coated polyimide substrate using indelible markers and an XY plotter. According to some embodiments, the frequency response window of the scattering parameter responses can be tuned by the resonator geometry. According to some embodiments, measuring the resonance frequncy in a range of from about 1 to about 100 MHz provides a clean spectral background and sufficient signal penetration through a medium such as soil and water. According to some embodiments, the activity of a hydrolytic enzyme can be measured by coating the surface of the resonator with a specific substrate to the enzyme and observing the degradation rate of the substrate as transduced wirelessly by a change in resonant frequency. According to some embodiments, resonance frequncy data can be fit with a custom transport, reaction model to extract the catalytic activities (e.g., turnover rate or k_{cat}) for an enzyme. According to some embodiments, the resonators can be used to transduce enzyme activity in closed environments. According to some embodiments the resonators can be deloyed to provide real-time in situ measurements of enzymatic activity. According to some embodiments, the resonator can be a an open circuit as opposed to a closed circuit.

### BRIEF DESCRIPTION OF THE FIGURES

The drawings illustrate generally, by way of example, but not by way of limitation, various embodiments discussed in the present document.

FIG. 1 is a sectional view of a resonator.

FIG. 2 is a top view of an electronically conductive segment broken away from the resonator of FIG. 1.

FIG. 3 is a plan view of a resonator reader.

FIG. 4 shows a fabrication method and method of use of the resonator according to an Example of the present disclosure

FIG. 5A is a schematic diagram showing fabrication and consumption of a substrate for an enzyme according to an Example of the present disclosure.

FIG. 5B shows a resonator S21 magnitude scattering parameter of the resonator of FIG. 5A.

FIG. 6A is a schematic diagram of a resonator according to an Example of the present disclosure.

FIG. 6B is a graph showing empirical determination of the correlation for frequency response to a change in radius of the resonator in FIG. 6A as the substrate is digested.

FIG. 6C is a graph showing the data of FIG. 6B, converted to a reactive front radius as a function of time using Matlab image processing.

FIG. 6D shows the frequency drop of the resonant sensor of FIG. 6A expressed as a function of the reactive radius location.

FIG. 6E shows the resonant sensor's radial response trajectory.

FIG. 6F shows the resonant sensor's turnover rate.

FIGS. 7A-7F are a set of graphs showing changes in resonant frequency in various resonators according to an Example of the present disclosure.

FIGS. 8A-8C are a set of graphs showing the effect of the composition of the substrate on resonant frequency of the resonator according to an Example of the present disclosure.

FIGS. 9A-9C is a set of graphs and pictures showing a resonator deployed in soil and graphs showing the resonance frequency of the resonator placed in soil according to an Example of the present disclosure.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 9%, 99.5%, 99.9%, 99.99%⁰, or at least about 99.999% or more, or 100%.

The term “organic group” as used herein refers to any carbon-containing functional group. Examples can include an oxygen-containing group such as an alkoxy group, a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, or cycloalkylalkyl. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, and the like; and R₃N wherein each R is independently selected, such as trialkylamines, dialkylarylamines, alkyldiarylamines, triarylamines, and the like. The term “amine” also includes ammonium ions as used herein.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “weight-average molecular weight” as used herein refers to M_{w}, which is equal to ΣMᵢ²nᵢ/ΣMᵢnᵢ, where nᵢ is the number of molecules of molecular weight Mᵢ. In various examples, the weight-average molecular weight can be determined using light scattering, small angle neutron scattering, X-ray scattering, and sedimentation velocity.

As used herein, the term “polymer” refers to a molecule having at least one repeating unit and can include copolymers.

The polymers described herein can terminate in any suitable way. In some embodiments, the polymers can terminate with an end group that is independently chosen from a suitable polymerization initiator, —H, —OH, a substituted or unsubstituted (C₁-C₂₀)hydrocarbyl (e.g., (C₁-C₁₀)alkyl or (C₆-C₂₀)aryl) interrupted with 0, 1, 2, or 3 groups independently selected from —O—, substituted or unsubstituted —NH—, and —S—, a poly(substituted or unsubstituted (C₁-C₂₀)hydrocarbyloxy), and a poly(substituted or unsubstituted (C₁-C₂₀)hydrocarbylamino).

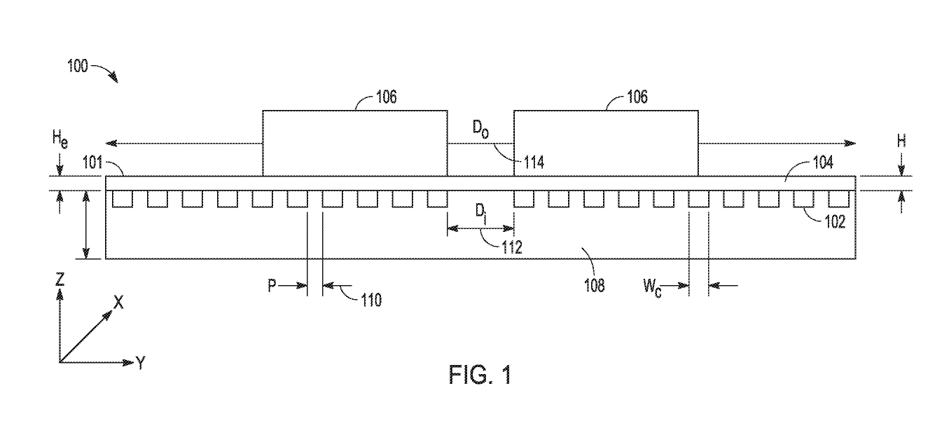

Described herein is a resonator for detecting enzymatic activity. Enzymatic activity can include the presence of an enzyme, a rate of reaction between an enzyme and a substrate, or other parameters. FIG. 1 is a sectional view of resonator system 100. Resonator system 100 includes resonator 101 including electronically conductive segment 102, polymeric component coating 104, substrate 106, and dielectric material 108.

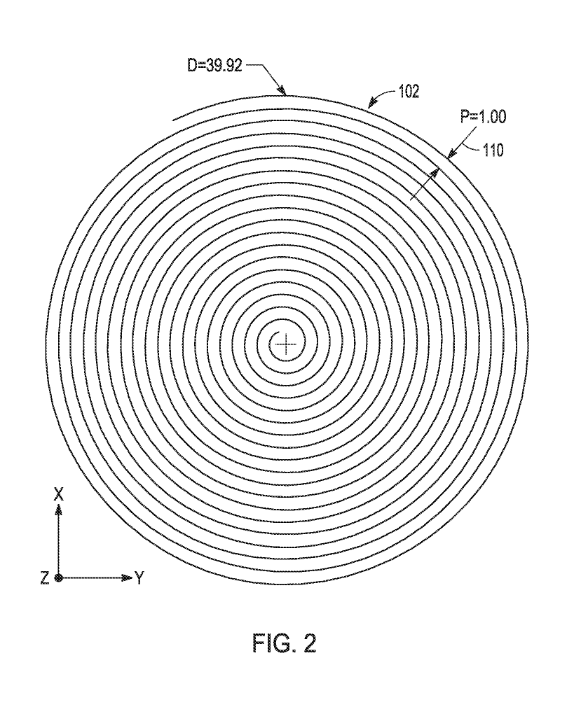

Electronically conductive segment 102 includes an electronically conductive metal. FIG. 2 is a top view of electronically conductive segment 102 broken away from the other components of resonator 101 shown in FIG. 1. As shown in FIG. 2, electronically conductive segment 102 includes a plurality of rings. Examples of suitable metals forming electronically conductive segment 102 include copper, silver, gold, aluminum, alloys thereof, or mixtures thereof. Electronically conductive segment 102 can be formed as a continuous segment or may include a plurality of discontinuous segments distributed through resonator 101. Electronically conductive segment 102 can take on any suitable shape or configuration. For example, electronically conductive segment 102 can be configured as a spiral in which adjacent portions or rings of electronically conductive segment 102 are spaced relative to each other defining a pitch therebetween. In some examples, the pitch can be constant across electronically conductive segment 102, thus, as shown, the spiral is an Archimedean spiral.

As shown in FIGS. 1 and 2, electronically conductive segment 102 is continuous. Electronically conductive segment 102 can have any suitable dimensions. For example, a total length of electronically conductive segment can be in a range of from about 5 mm to about 2000 mm, about 15 mm to about 40 mm, about 20 mm to about 25 mm, or less than, equal to, or greater than about 5 mm, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or about 2000 mm. In general, increasing the length of electronically conductive segment 102 decreases the resonance frequency of resonator 101. In embodiments of electronically conductive segment 102, such as that shown in FIGS. 1 and 2, where electronically conductive segment is a spiral the length refers to the total distances measured along electronically conductive segment from end to end.

A distance between opposed faces of adjacent portions of electronically conductive segment 102 is characterized as pitch 110. In some embodiments of conductive segment 102, pitch 110 is constant across all portions. In other embodiments, pitch 110 can be variable. In further embodiments, a first plurality of pitches 110 may be constant while a second plurality of pitches 110 may be variable. For example, as shown in FIG. 1, pitch 110 is less than pitch 112. At each instance, pitch 110 or 112 can be in a range of from about 0.1 mm to about 10 mm, about 1 mm to about 3 mm, or less than, equal to, or greater than about 0.1 mm, 0.5, 1, 1.5, 2, 2.5, 3, 3.5, 4, 4.5, 5, 5.5, 6, 6.5, 7, 7.5, 8, 8.5, 9, 9.5, or about 10 mm. Generally, increasing pitch 110 or 112 increases the resonance frequency of resonator 101.

As shown in FIGS. 1 and 2, resonator 101 has a circular profile. In other embodiments, however, resonator 101 can have any other suitable shape. For example, resonator 101 can have a polygonal profile such as a triangular shape, a square shape, a rectangular shape, a pentagonal shape, a hexagonal shape, a heptagonal shape, or an octagonal shape. A major dimension D₀ in the x-direction or y-direction that is perpendicular to electronically conductive segment 102 can be represented as a diameter or width of resonator 101. The major dimension can be in a range of from about 5 mm to about 100 mm, about 15 mm to about 60 mm, or less than, equal to, or greater than about 5 mm, 10, 15, 20, 25, 30, 35, 40, 45, 50, 55, 60, 65, 70, 75, 80, 85, 90, 95, or about 100 mm.

A height or thickness, measured in the z-direction, of electronically conductive segment 102 can be set to any value. For example, a thickness of electronically conductive segment 102 can be in a range of from about 10 μm to about 100 μm, about 20 μm to about 40 μm, or less than, equal to, or greater than about 10 μm, 15, 20, 25, 30, 35, 40, 45, 50, 55, 60, 65, 70, 75, 80, 85, 90, 95, or about 100 μm. The thickness of electronically conductive segment 102 can affect the resonance frequency of resonator 101. Increasing the thickness of electronically conductive segment 102 too much, however, can result in resonator 101 being too thick for certain applications.

Polymeric component 104 is located between electronically conductive segment 102 and substrate 106. Polymeric component 104 coats a portion of the total surface area of electronically conductive segment 102. For example, polymeric component 104 coats from about 10 percent surface area to about 70 percent surface area of the electronically conductive segment 102, about 20 percent surface area to about 33 percent surface area, or less than, equal to, or greater than about 10 percent surface area, 15, 20, 25, 30, 35, 40, 45, 50, 55, 60, 65, or about 70 percent surface area. Polymeric component can have a height or thickness in the z-direction that can be in a range of from about 10 μm to about 100 μm, about 20 μm to about 40 μm, or less than, equal to, or greater than about 10 μm, 15, 20, 25, 30, 35, 40, 45, 50, 55, 60, 65, 70, 75, 80, 85, 90, 95, or about 100 μm.

Polymeric component 104 can include an electronically insulating material. For example, polymeric component 104 can include a polyimide. Where present, the polyimide can include a repeating unit having the structure according to Formula I:

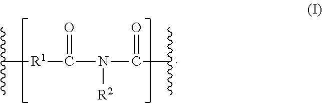

In Formula I, R¹ can be chosen from —O—, —NH—, and substituted or unsubstituted (C₁-C₂₀)hydrocarbylene. Additionally, R² can be chosen from —H, —OH, and substituted or unsubstituted (C₁-C₂₀)hydrocarbyl. In some embodiments the (C₁-C₂₀)hydrocarbylene can be chosen from (C₁-C₂₀)alkylene, (C₁-C₂₀)alkenylene, (C₁-C₂₀)cycloalkylene, and (C₁-C₂₀)arylene. Additionally, in some embodiments, the (C₁-C₂₀)hydrocarbyl can be chosen from (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, and (C₁-C₂₀)alkoxy.

According to further embodiments, the polyimide can include a repeating unit having the structure according to Formula II:

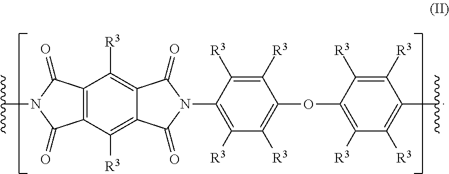

At each occurrence, R³ can be chosen from —H, —OH, and substituted or unsubstituted (C₁-C₂₀)hydrocarbyl. In some embodiments the (C₁-C₂₀)hydrocarbylene can be chosen from (C₁-C₂₀)alkylene, (C₁-C₂₀)alkenylene, (C₁-C₂₀)cycloalkylene, and (C₁-C₂₀)arylene. Additionally, in some embodiments, the (C₁-C₂₀)hydrocarbyl can be chosen from (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, and (C₁-C₂₀)alkoxy.

Other examples of suitable polyimides include a polyimide sold under the tradename KAPTON sold by DuPont of Wilmington Del. or a polyimide sold under the tradename PYRALUX sold by DuPont of Wilmington Del. The specific structure of the polyimide will depend on whether it may be stable during screen printing or etching and whether the polyimide is sufficiently insulative so that the resonator does not short during operation.

The portion of electronically conductive segment 102 that is free of contact with polymeric component 104 can be coated with dielectric layer 108. Dielectric layer 108 can have a height or thickness in the z-direction that is equal to or greater than that of polymeric component 104. For example, the thickness can be in a range of from about 0.30 mm and about 2 mm, about 0.90 mm to about 1.1 mm, or less than, equal to, or greater than about 0.30 mm, 0.35, 0.40, 0.45, 0.50, 0.55, 0.60, 0.65, 0.70, 0.75, 0.80, 0.85, 0.90, 0.95, 1, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95 or about 2 mm. Additionally, a major dimension such as a width in the x-direction or y-direction can be the same as a width in the x-direction or y-direction as polymeric component 104. Dielectric layer 108 can shield electronically conductive segment 102 and can include any suitable dielectric material. Examples of suitable dielectric materials include a bismaleimide-triazine (BT) resin, an epoxy resin, a polyurethane, a benzocyclobutene (BCB), a high-density polyethylene (HDPE), and combinations thereof. In some examples, the dielectric material can be air that can be located within a chamber enclosing the portion of electronically conductive segment 102 that is free of contact with polyimide coating 104.

Substrate 106 is in direct contact with polymeric component 104. Substrate 106 can be a substrate of one or more enzymes. For example, substrate 106 can be a substrate of a hydrolase enzyme (alternatively known as a EC 3 enzyme). The hydrolase can be classified by the bond it acts upon. For example, the hydrolase can be chosen from an esterase, nuclease, phosphodiesterase, lipase, phosphatase, DNA glycosylase, glycoside hydrolase, proteases, peptidase, acid anhydride hydrolase, helicase, GTPase, alcalase, or mixtures thereof. The enzyme can be present in system 100 or may be an external component that interacts with system 100. Additional enzymes may include a ligase and a lyase.

Substrate 106 can be a freestanding structure on polymeric component 104. Alternatively, substrate 106 can be disposed within a chamber located on polymeric component 104. The chamber can be open ended to allow substrate 106 to be exposed to an enzyme. In some examples, the chamber can be an O-ring. Substrate 106 can include cavity 114. Cavity 114 can extend in the z-direction to any suitable depth. For example, cavity 114 can extend from an exposed surface of substrate 106 to a surface of polymeric component 104.

Substrate 106 can be a substrate for any predetermined enzyme. That is, substrate 106 can be a substrate of the enzyme to which resonator system 100 is configured to detect enzymatic activity. In some embodiments, substrate 106 is a substrate of a hydrolase enzyme. Where the substrate 106 is a substrate of a hydrolase, substrate can include a bond that is hydrolyzable by the hydrolase. Examples of such bonds can include an ester bond, a glycosylic bond, an ether bond, a peptide bond, an acid anhydride bond, a halide bond, a phosphorous-sulfur bond, a sulfur-sulfur bond, a carbon-phosphorous bond, a carbon-sulphur bond, or a combination thereof. Examples of suitable substrates include a bovine serum albumin, citrus pectin, carboxymethyl cellulose, gelatin, polylactic acid, or a mixture thereof.

Resonator system 100 can further include resonator reader 200. FIG. 3 is a plan view of resonator reader 200. As shown in FIG. 3 resonator reader 200 includes first electronically conductive loop 202 and second electronically conductive loop 204. Resonator reader 200 further includes first connector 206 and second connector 208. First electronically conductive loop 202 and second electronically conductive loop 206 can independently include an electronically conductive metal such as copper, silver, gold, aluminum, alloys thereof, or mixtures thereof. A diameter of each of loops 202 and 204 can independently range from about 5 mm to about 100 mm, about 15 mm to about 60 mm, or less than, equal to, or greater than about 5 mm, 10, 15, 20, 25, 30, 35, 40, 45, 50, 55, 60, 65, 70, 75, 80, 85, 90, 95, or about 100 mm. As shown in FIG. 3, loops 202 and 204 overlap. A distance between overlapping regions of reader 200 can be in a range of from about 5 mm to about 100 mm, about 15 mm to about 60 mm, or less than, equal to, or greater than about 5 mm, 10, 15, 20, 25, 30, 35, 40, 45, 50, 55, 60, 65, 70, 75, 80, 85, 90, or about 95.

Connectors 206 and 208 can connect resonator reader 200 to a component such as a vector network analyzer. The vector network analyzer can in turn be connected to a computer. The connection between the vector network analyzer and the computer can be through a wire or an antenna. Loops 202 and 204 as well as connectors 206 and 208 are at least partially enclosed by a dielectric material. Examples of suitable dielectric materials include a polyimide, a bismaleimide-triazine (BT) resin, an epoxy resin, a polyurethane, a benzocyclobutene (BCB), a high-density polyethylene (HDPE), and combinations thereof.

Resonator reader 200 can be positioned substantially in line with resonator 101. A distance between resonator reader 200 and resonator 101 can be varied to improve performance. For example, a distance between resonator reader 200 and resonator 101 can be in a range of from about 1 mm to about 10 cm.

Resonator system 100 is described as including one resonator 101 and one resonator reader 200. However, in further embodiments resonator system 100 can include any plural number of resonators 101 and readers 200. In embodiments that include multiple resonators, each resonator can be designed to have a different initial resonant frequency. This can be accomplished by varying any parameter such as respective lengths of electronically conductive segments 102 or altering pitches 110 and 112. The resonators can also differ by the composition of the respective substrates. For example, a substrate of one substrate can be a substrate of a first enzyme while another substrate on another resonator may be a substrate for a different enzyme.

In operation, detecting enzymatic activity or the presence of an enzyme using resonator system 100 includes measuring a first resonance frequency of resonator 101. The first resonance frequency is the resonance frequency of resonator 101 before substrate 106 is contacted with an enzyme. When the enzyme is contacted with substrate 106, substrate 106 is consumed. If the enzyme is placed in cavity 114, substrate 106 is consumed in a radial direction. As substrate 106 is consumed, the resonance frequency of resonator 101 changes. Thus, if a second resonance frequency of resonator 101 is measured that is different than the first resonance frequency the presence of the enzyme can be confirmed. By measuring the rate of change of the resonance frequency it is possible to monitor the rate of reaction between substrate 106 and the enzyme. At least one of the first resonance frequency and the second resonance frequency can be in a range of from about 1 MHz to about 500 MHz, about 1 MHz to about 100 MHz, or less than, equal to, or greater than about 1 MHz, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or about 500 MHz.

In embodiments where resonator system 100 includes multiple resonators 101, each resonator 101 may have different substrates 106 for different enzymes. Each resonator can be configured to have a different first resonance frequency. Therefore, specific substrates 106 for predetermined enzymes can be paired with resonators having specific known first resonance frequencies. If the resonance frequency of one of resonators 101 begins to change then the presence of a specific enzyme and the absence of another can be confirmed.

Resonator system 100 can be deployed in many different mediums to detect enzymatic activity. For example, resonator system 100 can be placed in soil, fabric, or a tank. If placed in soil, resonator system 100 can detect the presence of certain harmful or beneficial enzymes that can impact the viability of crops. If placed in a tank, resonator system 100 can be used to detect enzymes in a storage tank used for example to store chemicals, beverages, medicine, or drinking water. The tank can also be a component of a bioreactor. The presence of the enzyme may indicate that the solution stored in the tank is not safe for consumption. Alternatively, if the presence of a certain enzyme is desirable, then the levels of the enzyme can be monitored. If resonator system 100 is placed in fabric, then it can be possible to determine whether the fabric is exposed to a biological agent. For example, resonator system 100 can be placed in a garment of a military member or first responder to allow them to know in real time whether they have been exposed to a biological agent.

Resonator system 100 can be assembled according to any suitable method. For example, an assembly including any of the electronically conductive metals described herein coated to the material of the polymeric component can be etched to form the pattern (e.g., the spiral) of electronically conductive segment 102. To form the pattern the electronically conductive metal can have the pattern printed on the surface to effectively block some portions of the electronically conductive metal from the etchant. The pattern can be printed with an indelible marker.

Etching can include at least partially immersing the electronically conductive segment in a solution comprising an etchant. The etchant can include any solution capable of etching the electronically conductive metal but not the polymeric component. As an example, the etchant can include hydrogen peroxide and hydrochloric acid.

Following etching the substrate is contacted with polymeric component 104. The material forming substrate 106 can be applied as a liquid and at least partially solidified thereon. Substrate 106 can be directly applied to polymeric component 104. In some embodiments, a chamber can be placed on polymeric component 104 and the material of substrate 106 can be deposited therein. After substrate 106 is formed, cavity 114 is formed in the center of substrate 106. The enzyme of a solution including the enzyme can be placed within cavity 114.

### Examples

Various embodiments of the present disclosure can be better understood by reference to the following Examples, which are offered by way of illustration. The present disclosure is not limited to the Examples given herein.

Bacterial protease, gelatin, and hydrogen peroxide (H₂O₂, 3%) were purchased from Carolina Biological Supply Company. Hydrochloric acid (HCl) and acetone (C₃H₆O) were procured from Fisher Scientific. Protease from Bacillus licheniformis (Subtilisin A, 7-15 units/mg solid) and red fluorescent amine-modified polystyrene latex beads (aqueous suspension, 1.0 μm mean particle size) were bought from Sigma-Aldrich and glycerol (C₃H₈O₃) was purchased from VWR. Dupont Pyralux® AC flexible sheet and a Silhouette Curio XY plotter were utilized for resonator fabrication. An S5048 two-port vector network analyzer from Copper Mountain Technologies was used to measure the resonant sensor scattering parameters. This was coupled to a two loop antenna constructed in a custom, 3D-printed housing using an Airwolf Axiom 3D printer. The 3D printer was also used to print resonant sensor cases for soil testing. A Logitech C920 HD Pro Webcam was used for time-lapse photography.

Spirals with different pitch (1-2.5 mm) and lengths (407-2764 mm) which resulted in resonators of outer diameters 15-60 mm) sizes were designed using Rhino 5 software. Using the Silhouette CURIOprinter with a Sharpie ultra fine point permanent marker, spirals were drawn on Dupont PYRALUX printed circuit board flexible polyimide (25 μm) with a thin layer of copper (35 μm). The Pyralux sheet was subsequently etched using a solution of hydrogen peroxide and hydrochloric acid with a 2:1 ratio (e.g., 15 ml of HCl was added to 30 ml of H₂O₂ for each resonator) in the fume hood. The optimal etching time to prevent over-etching of the masked spiral trace while removing the undesired copper was found to be 15 to 25 min depending on the size of the resonator. The etched Pyralux was then rinsed with acetone to remove the permanent marker. The flexible resonator was then inverted and epoxied on a petri dish to protect the resonator from shorting; in this mode, the polyimide film becomes the sensor surface.

The resonant frequency of the resonator of the instant example was measured using two copper loop antennas with a diameter of 54 mm and overlap of 26.7 mm set in a 3D-printed reader frame; this was connected to a two-port vector network analyzer (VNA). For studying the response of this class of resonant-based sensors, the magnitude of S21 in a frequency range of 1-100 MHz was monitored. The standard resonator geometry used in the measurements was the spiral with an outer diameter of 40 mm, an inner diameter of 1.5 mm, and a pitch of 1 mm having a resonant frequency of approximately 75 MHz in the air. The copper wires were soldered to a BNC plug and mutually grounded to minimize the effects of cable length and position. These plugs were then connected to the VNA ports using a shielded and immobilized set of BNC cables. The mutual inductance coupling between the resonant sensor and the reader coils was sufficiently strong to provide a clear S21 signal up to a 5 cm stand off distance in the air. The VNA was connected to a Lenovo laptop with Windows OS and the VNA data acquisition was automated via Matlab. The resonant frequency was identified as the peak of the characteristic S21 magnitude sigmoidal response.

Gelatin (3.5 to 15 g) per 100 ml DI water and 0 to 30 g plasticizer (glycerol) per 100 g gelatin were used for preparing different gelatin substrates and finding a sensor coating that was structurally robust yet had a signal response in an hour time frame for these studies. To make the gelatin substrate, DI water was preheated to 72° C. using a water bath on a hot plate. Granular gelatin procured from Carolina was added to the water and gently stirred using a stir bar. When the bath's temperature raised to 80° C., glycerol was added and the mixture was stirred for 10 minutes. The resonant sensor starting frequency and extent of signal modulation were affected by the surface area of substrate added. When an O-ring with a 12.5 mm diameter was epoxied at the center of the 40 mm resonator, the resulting center well surface area was found to have a clear start resonant frequency and signal response in the desired 1-100 MHz range. A 200 μl volume of substrate solution was poured into the center of the O-ring and cured for two hours at room temperature. Afterward, a 7.56 mm diameter hole was made at the center of the solidified gelatin substrate to serve as the enzyme addition well.

For the empirical relation of resonant frequency to radius measurement used to inform the transport model of the enzyme-gelatin system, 15 μl of red fluorescent amine-modified polystyrene latex beads aqueous suspension was added to 15 μl of 400 mg/ml solution of bacterial protease in PBS. This solution was poured into the gelatin hole and the webcam setup was used to take images of the degradation extent of every S21 data acquisition point.

The Petri dish was fixed above the reader coils at a 5 mm displacement and the S21 magnitude (dB) versus the frequency (MHz) was collected every 15 seconds using automated Matlab scripts. After three minutes, 30 μl of the enzyme solution was pipetted into the cut center well. The hydrogel degradation was monitored for three hours. The enzyme solutions used for these tests were prepared by dissolving 15 to 200 mg of either bacterial protease or subtilisin A in 1 ml phosphate-buffered saline (PBS) with a pH of 7.95. All tests in this Example were run at room temperature.

Soil samples were collected from the field and stored at −80° C. prior to testing. For these tests, the hydrogel was made using 7 wt % gelatin and 20 g glycerol/100 g gelatin. 200 μl of the gelatin solution was placed at the center of the epoxied O-ring on the standard resonator (Dₒ=40 mm, P=1 mm). After solidification, the gelatin was covered with parafilm and a 7.56 mm diameter hole was made at the center of the gelatin and parafilm. After saving the background signal for two minutes, 1.1 g of previously frozen non-sandy soil was premixed with 300 μl DI water and placed in the center, sample hole. Wet paper towels were placed around the resonator in a petri dish to minimize evaporation over the course of the experiment.

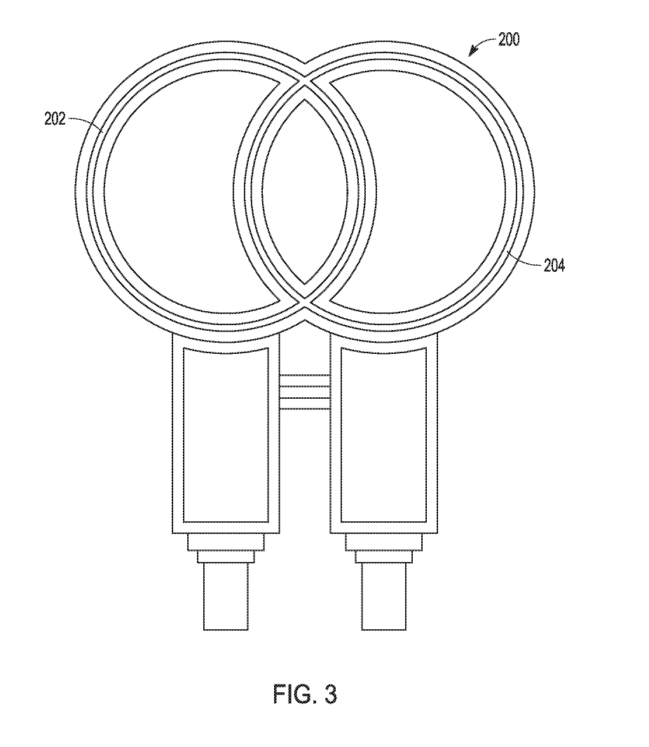

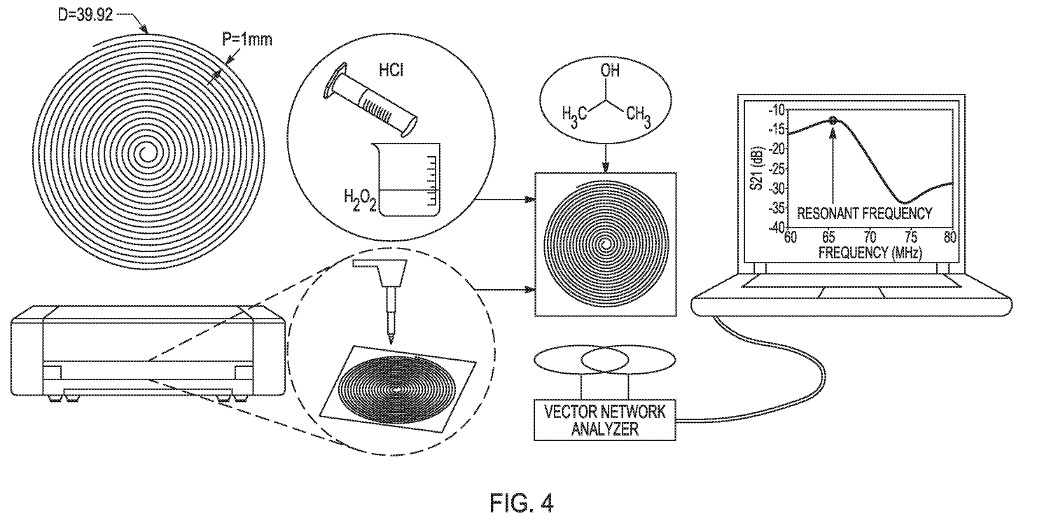

With initial prototyping, it was found that prior resonant sensor designs that relied on a rigid closed loop circuit or parallel plates folded together to complete a circuit, could be greatly simplified as an open circuit Archimedean coil. In this design, the conductive spiral serves as the inductor and the narrow gaps between the conductive lines operate as the tuning capacitor. Resonators of varying lengths and pitch sizes were fabricated to determine the influence of resonator geometry on the start resonant frequency. A fabrication method and method of use of the resonator is shown in FIG. 4. To manufacture the resonator, a mask is drawn on the Pyralux® copper surface using an indelible marker and XY plotter. Unwanted copper is removed by etching. The marker was sufficient to protect the conductive trace with minimal under etching. This process allowed for rapid prototype development in our own lab with the trade-off being larger feature sizes (down to 200 μm can be resolved). Smaller features can be made via screen printing or lithographic mask techniques, albeit at a longer lead time between new designs. The resonator attached to a vector network analyzer outputs data to a device such as a laptop. Although shown as a wired connection, the connection between the vector network analyzer and device can also be wireless.

In order to determine the effect of resonator length, multiple resonators with constant pitches of 1.2 mm and different lengths (407-2764 mm) were fabricated. The resonant frequency of the resonators showed an inverse relationship with the length. Next, to study the effect of the pitch size, five resonators with a set length of 1255 mm were designed with varying pitch sizes of 1, 1.2, 1.6, 2, and 2.5 mm. The resonant frequency of the resonator has a direct relationship with the pitch size (e.g., resonant frequency increased with increasing pitch size. The basic governing equation for the resonant frequency of an LC circuit with a parallel plate capacitor is as follows:

$$\frac{1}{2 π \sqrt{\frac{L {ɛ}_{r} {ɛ}_{0} A}{d}}} \qquad ( Eq . 1 )$$

where f is the resonant frequency, L is the inductance, εᵣ is the relative permittivity of the material, ε₀ is the permittivity of free space and (a constant equal to 8.854*10⁻¹² F/m), A is the capacitive area, and d is the capacitor plate displacement. Thus, the empirical observations of geometric effects on starting resonant frequency are physically grounded, as the increase in coil length results in an increased inductance and the increased pitch size decreases the capacitance.

This governing equation (eq. 1) also shows that the relative permittivity of the material near the inductive coils also has a direct effect on the resonant frequency. This was validated by observing the change in resonant frequency of a sensor going from an air relative permittivity (εᵣ≈1), to placing water (εᵣ≈80 at 20° C.) on the sensor surface and then replacing the water with oil (εᵣ≈3 at 20° C.). Each caused an expected shift based on the change in the dielectric. Since the resonant frequency is inversely proportional to the permittivity, the resonant frequency of air started at a higher frequency in comparison with a high-dielectric medium of water. For a medium with intermediate permittivity (vegetable oil), the resonant frequency was between the water and air. Furthermore, it was determined that the resonant sensor was responsive to the amount of liquid present on the surface, by adding increasing amounts of deionized (DI) water and vegetable oil (0 to 4.5 ml). The change in resonant frequency for adding high relative permittivity water was 60 MHz while this shift was about 4 MHz for the same volume of oil.

With this fundamental understanding of resonator frequency tuning and response to a change in the surface dielectric, a sensor was designed that would transduce the activity of hydrolytic enzymes. By coating the polyimide surface with the target substrate of the enzyme, the resonator has a defined starting frequency; as the enzyme degrades the substrate, the change in the surface dielectric is then transduced via a change in the resonant frequency (much the same as shifting from oil to water as above). The optimal amount of substrate to place at the center of the resonator to allow for a full response window in the 1-100 MHz range. This frequency range was targeted for its ability to penetrate aqueous, tissue, and plastic systems.

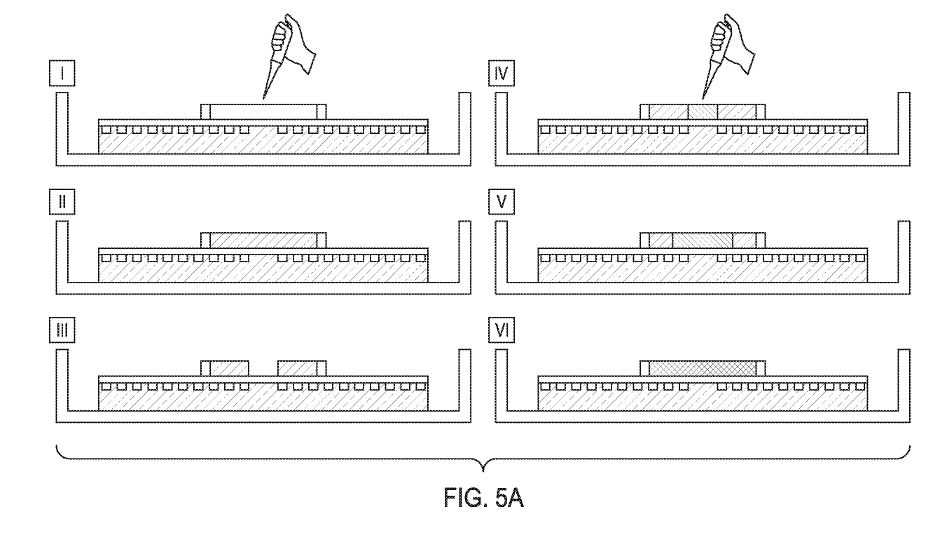

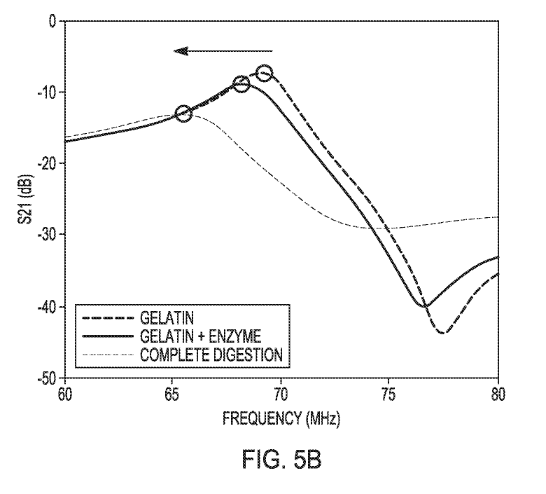

FIG. 5A schematically shows a method of manufactuing an assembly where a substrate is disposed on the resonator. To improve reproducibility of the tests, the assembly included an O-ring (12 mm ID) was secured to the center of the resonator in which the enzyme substrate could be drop cast (FIGS. 5A-I). For the tests in this Example, the substrate was gelatin and the hydrolytic enzymes were various proteases. The gelatin would set (FIGS. 5A-II) and a punch was used to remove a 4 mm diameter section of gel (FIGS. 5A-II) to create a test well to insert the enzyme (FIGS. 5A-IV). The resonator was then placed on a two loop antenna reader in order to study the substrate degradation process (FIGS. 5A-V and -VI). FIG. 5B. FIG. 5B shows the resonator S21 magnitude scattering parameter as observed. The resonant frequency is defined as the peak of the sigmoidal S21 curve.

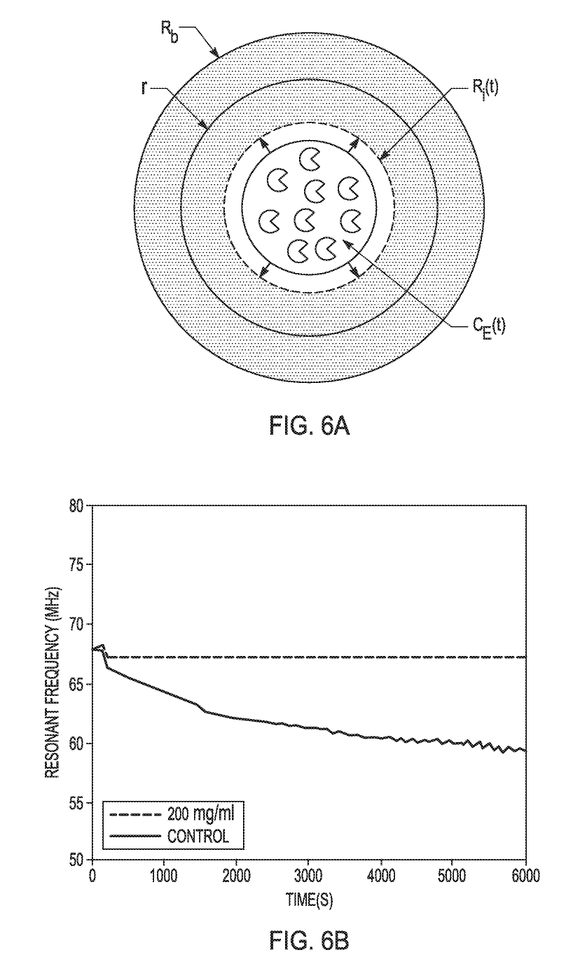

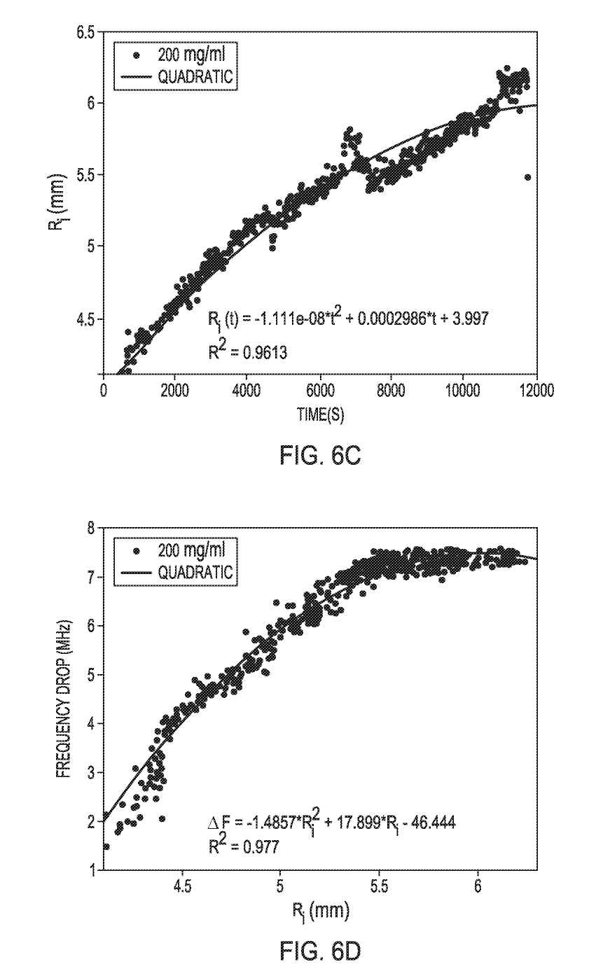

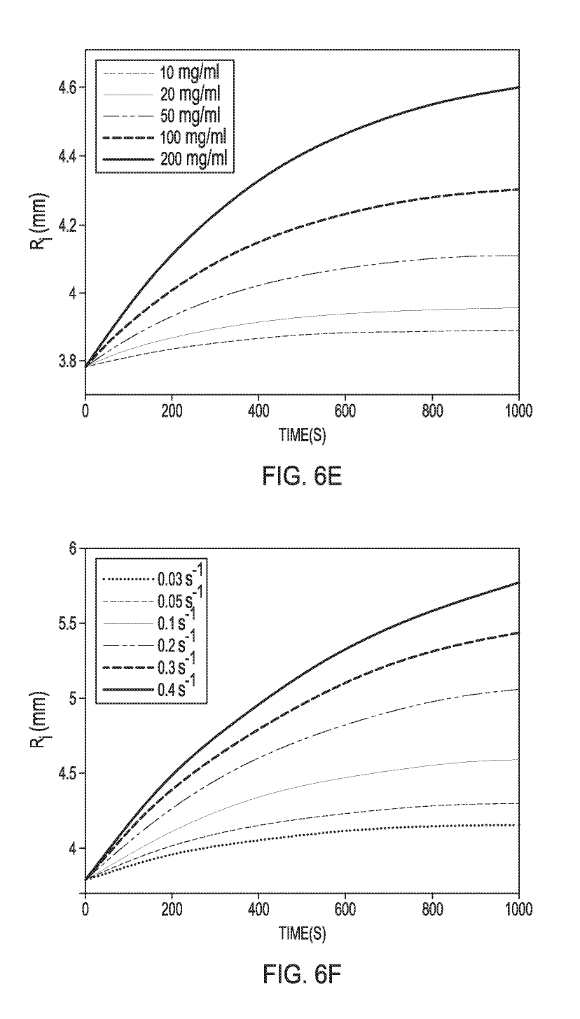

The radial gel digestion process was modeled to fit the frequency response data with an enzyme turnover rate (mass of substrate digested per mass of enzyme for a given unit of time). The model geometry (FIG. 6A) readily lends itself to the cylindrical coordinate version of conservation of mass transport equation.³⁷

$$\frac{δ C}{δ t} + {v}_{r} \frac{δ C}{δ r} + \frac{{v}_{θ}}{r} \frac{δ C}{δ θ} + {v}_{z} \frac{δ C}{δ z} = D [ \frac{1}{r} \frac{δ}{δ r} ( r \frac{δ C}{δ r} ) + \frac{1}{{r}^{2}} \frac{{δ}^{2} C}{δ {θ}^{2}} + \frac{{δ}^{2} C}{δ {z}^{2}} ] + {R}_{v}$$

Where C is the concentration of enzyme in the system, v is the transport velocity, D is the diffusion of the enzyme through the gelatin hydrogel, R is the rate of enzyme depletion, and r, θ, and z are radial, angular, and height dimensions. By assuming the total enzyme concentration in the system is constant (R=0, no depletion) and simplifying the problem to a singular radial dimension, we obtain the following partial differential equation (PDE):

$$\frac{\partial C}{\partial t} = D [ \frac{1}{r} \frac{δ}{δ r} ( r \frac{δ C}{δ r} ) ]$$

This PDE is governed by the following boundary conditions: 1) at time zero, the enzyme concentration throughout the entire substrate gel is zero, 2) at all times the flux at the O-ring interface (r=R_{b} in FIG. 6A) is zero, and 3) the concentration of enzyme at the reactive interface (Rᵢ(t) in FIG. 6A) is equal to the concentration of enzyme in the center well. The third boundary condition is actually a Stefan boundary condition because its location is time-dependent (as the gel digests, the reactive interface moves towards the O-ring). The third boundary condition is further complicated because the center well enzyme concentration is also depleting with time due to diffusion of the enzyme into the gel. This problem cannot be solved analytically, and thus was modeled using numerical methods, namely centered finite differences in the spatial domain with a grid resolution of 0 μm and 4^{th} order Runge Kutta solver (Matlab ode45) in the time domain.

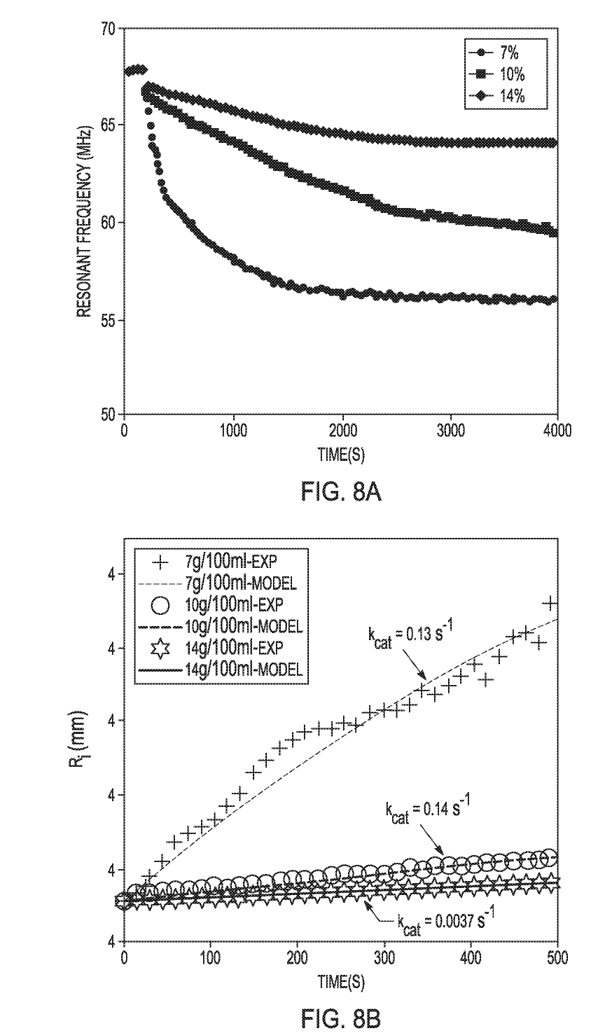

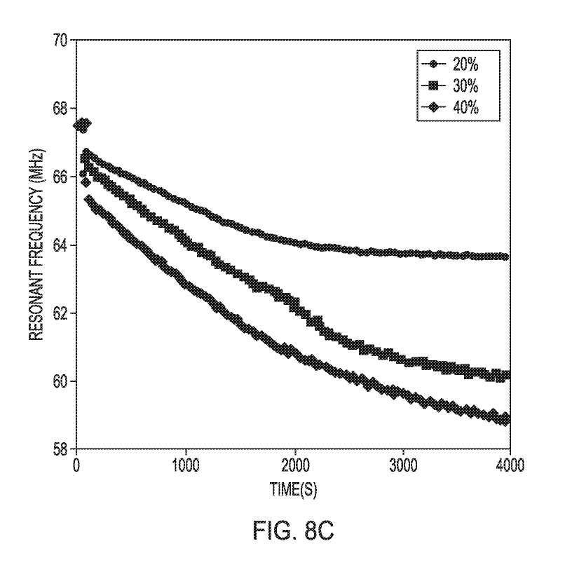

One missing link for the model is the correlation of frequency response to a change in radius. This was determined empirically by recording the motion of dyed polystyrene beads as the gel digests (FIG. 6B) which was converted to the reactive front radius as a function of time using Matlab image processing (FIG. 6C). The frequency drop of the resonant sensor can then be expressed as a function of the reactive radius location (FIG. 6D). The model's radial response trajectories are dependent on the enzyme start concentration (FIG. 6E), turnover rate (FIG. 6F), and diffusion constant (set at 10 cm²/s for these figures). For the diffusion constant literature values ranging from 1.017*10⁻⁶ to 2.46*10⁻⁶ cm²/s in water⁴¹⁻⁴³ and from 4*10⁻⁸ to 5*10⁻⁷ cm²/s in hydrogel (which is approximately 10 to 100 times lower in comparison with the diffusion coefficient in water) were found and the model at 1.7*10⁻⁷ cm²/s based on goodness of fit to the data. This diffusion constant is dependent on the substrate concentration and composition, as different best fit diffusion constants are present for gels at new gelatin and glycerol concentrations (FIG. 8).

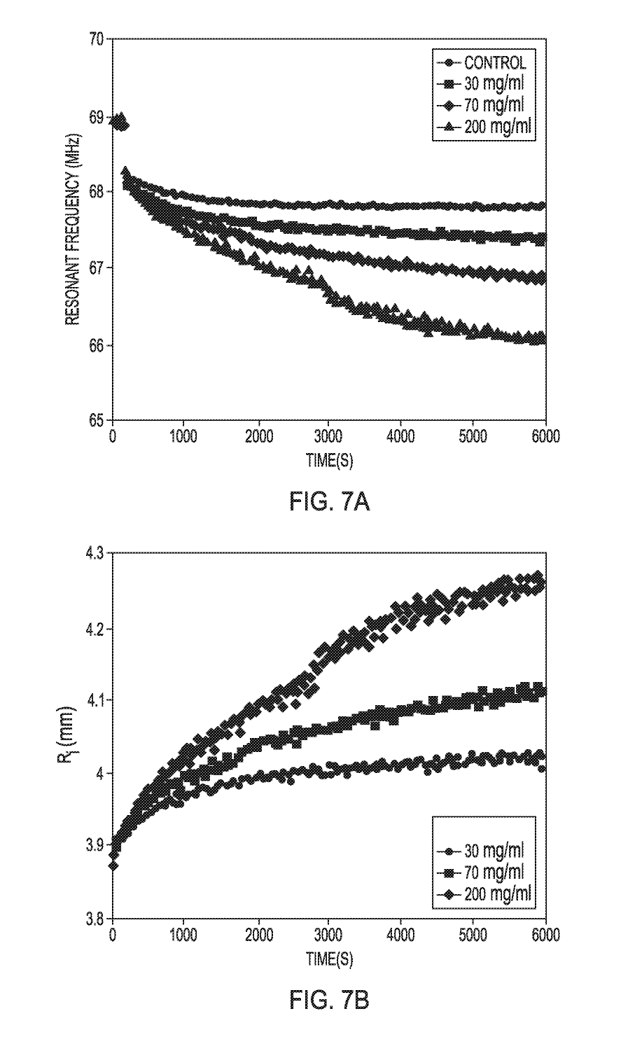

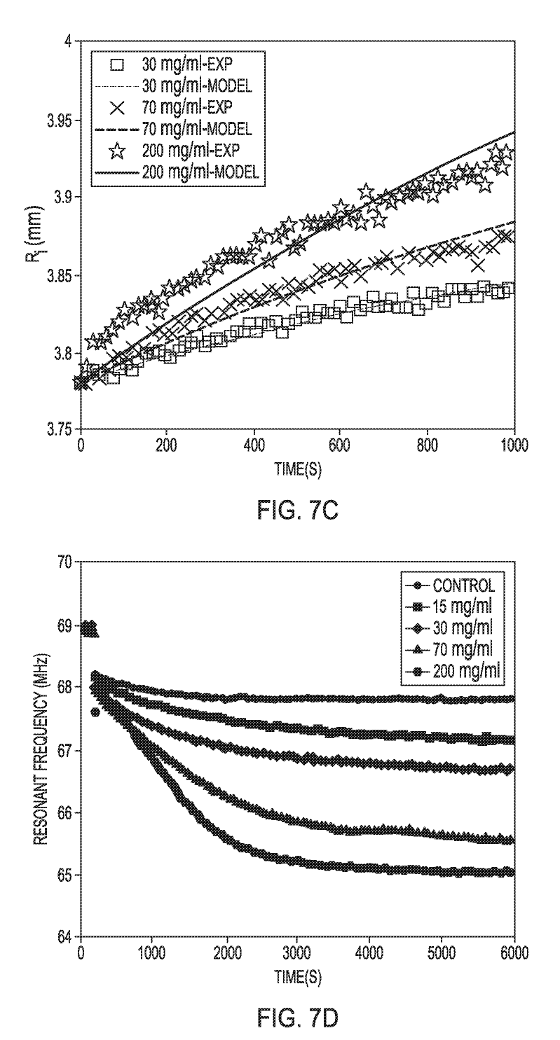

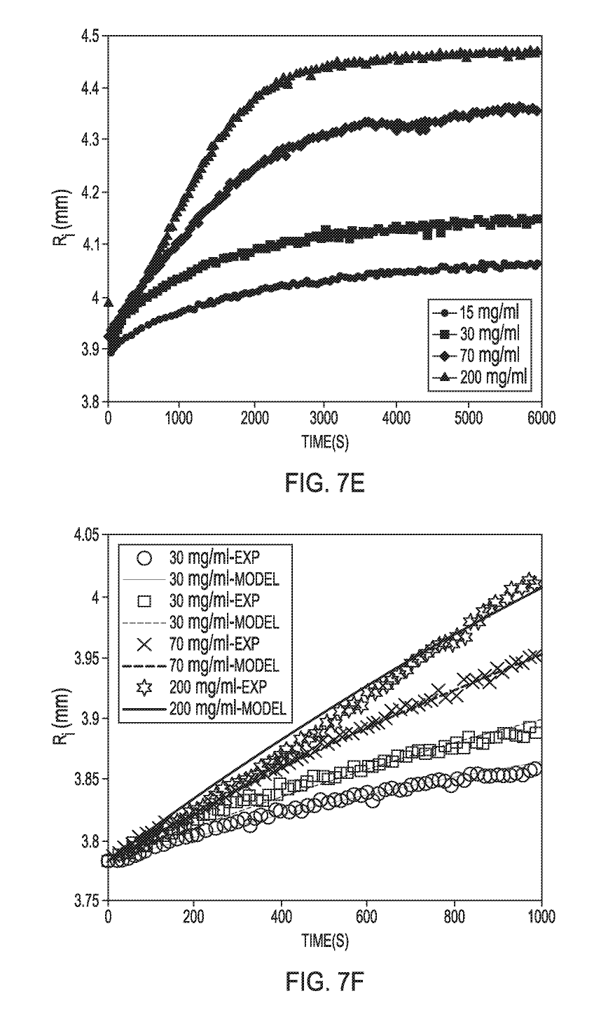

The effect of enzyme concentration on the resonant sensor response was studied for two proteases. The gelatin substrate composition for these tests was 14 wt % gelatin and 20 g glycerol/100 g gelatin. Proteinase K was tested at concentrations of 30, 70, and 200 mg/ml (FIG. 7a). Bacterial protease was tested at concentrations of 15, 30, 70, and 200 mg/ml (FIG. 7b). The S21 magnitude from 1 to 100 MHz was recorded for three hours after enzyme addition. The peak of the resonant sensor sigmoidal curve was then plotted as a function of time (FIG. 7c). The resonant frequency decreased for all enzyme concentrations. For all traces there was a large, initial decrement in resonant frequency due to the addition of liquid (high relative permittivity) to the dry resonator, however, after this initial jump, the rate of change was dependent on enzyme concentration, with the higher enzyme concentrations leading to lower resonant frequencies. This is due to a larger extent of digestion, as observed in the frequency drop vs. reaction radius experiment (FIG. 6c). As the gelatin degrades, there is a dielectric shift which we attribute to more water access on the resonator surface which causes the resonant frequency decrease.

Using the empirical relations herein, the resonant frequency vs. time data can then be translated to the position of the reactive front (Rᵢ) as a function of time (FIG. 7d). The model can then be used to determine the turnover rate (k_{cat}) that gives the best fit to the experimental data (FIG. 7e and Table 1). The model fit is constrained to the first 1000 s time points due to simulation time constraints (roughly 10 minutes per simulation which must be iterated many times to converge on best k_{cat} value). The km of an enzyme is an intrinsic property and thus should be conserved through the different enzyme concentrations. The small variations could be due to experimental variance, but it could also be due to the increased amount of enzyme present in a solution that acts as a competitive substrate. At higher enzyme concentrations, some of the enzymes will digest other enzymes, rather than the substrate, this would decrease the observed km. Comparison to literature values of enzyme activity is difficult, as these are typically defined with activity units (relative measures to another protease or known substrate analog). In this case, Subtilisin A activity is specified by the vendor (Sigma) as 7-15 units/mg solid in which the unit is defined as the production of 181 μg of tyrosine (conveniently measured by absorbance in simple samples) per minute by hydrolysis of casein at 37° C. and pH 7.5. Assuming 7 units/mg solid, the activity of subtilisin A provided by the vendor is 0.0211 s⁻¹ on a gram tyrosine per gram enzyme basis. This value is roughly comparable to the k_{cat} values obtained from the resonant sensor response using the model fit considering the fact that the experiments in this paper were conducted at 20° C. and we are measuring with reference to the start substrate mass (gelatin). The k_{cat} substrate basis is a more useful catalytic measure, as tyrosine is only one of many byproducts from digestion, and we can report activity on degradation of an actual substrate of interest (whatever is coated on the resonator).

TABLE 1
k_{cat} values (s⁻¹) used in the model fit for different concentrations
of subtilisin A and bacterial protease at D = 1.7*10⁻⁷ cm²/s.

| | Concentration | | | |
|---|---|---|---|---|
| Enzyme Type | 15 (mg/ml) | 30 (mg/ml) | 70 (mg/ml) | 200 (mg/ml) |
| Subtilisin A | — | 0.003 | 0.00275 | 0.00205 |
| Bacterial Protease | 0.009 | 0.008 | 0.0065 | 0.0037 |

In order to study the effects of hydrogel composition on the sensor response, substrates with different gelatin and glycerol concentrations were made and tested with 200 mg/ml bacterial protease solution. Hydrogels containing 7, 10, and 14 wt % gelatin with constant plasticizer concentration (20 g glycerol/100 g gelatin) were measured. The drop in resonant frequency showed an inverse relationship with the gelatin concentration (FIG. 8a). The digestion model was run at these higher gelatin concentrations in order to predict the enzyme activity for these data (FIG. 8b) and resulted in k_{cat} fit values of 0.13, 0.014, and 0.0037 s⁻¹ for 7, 10, and 14 wt % gelatin respectively. To get best fit for 7 wt % gelatin, we had to increase the diffusion constant to 10⁶ cm²/s, which is physically reasonable as a decreasing amount of gelatin would have larger pore sizes and an increased diffusion constant. It is hypothesized that the observed decrease in k_{cat} as the gelatin concentration increases is a real effect due to different structural morphologies of the gelatin (more entangled structures at higher concentrations vs. easier to digest, sparse structures at lower concentrations). For studying the effect of plasticizer, 20, 30, and 40 g glycerol/100 g gelatin were used in a 14 wt % gelatin hydrogel. It was seen that higher plasticizer concentration led to a lower resonant frequency (FIG. 8c). This was atributed to a higher swelling ratio, allowing for more diffucsion of enzyme, and thus greater rate of digestion. In effect, these experiments demonstrate the tunability of the sensor response; the response rate and signal duration can be tuned for target enzyme concentrations based on substrate composition.

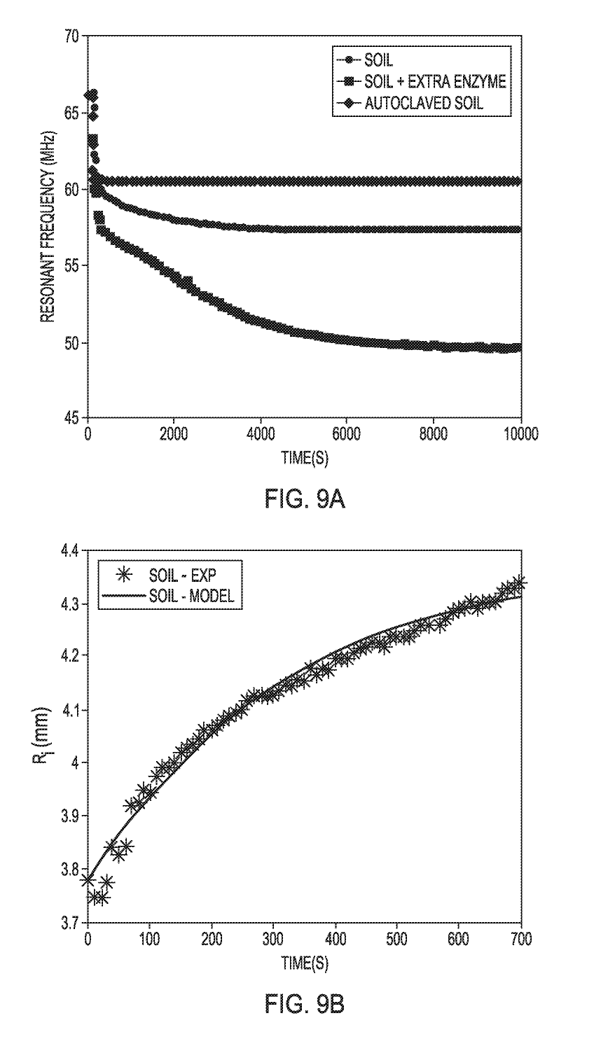

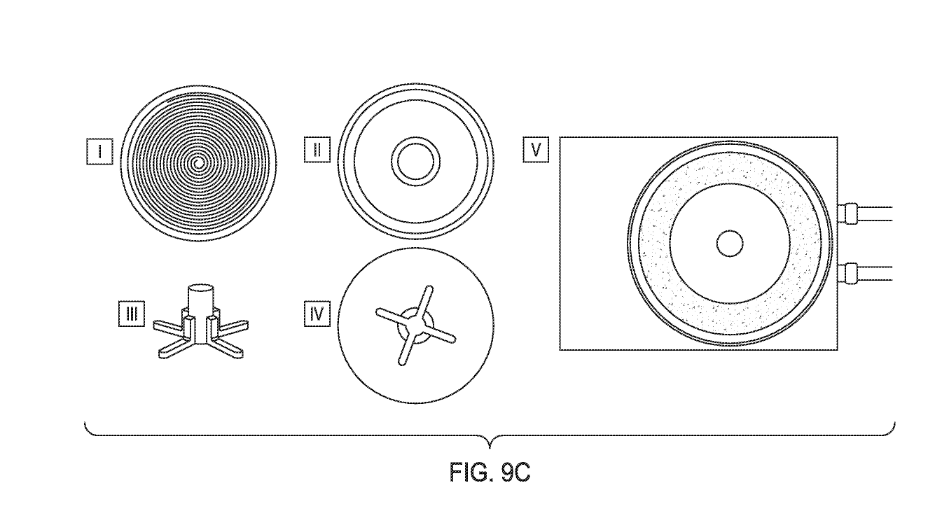

To test the capability of the resonant-based sensor for contact free, in situ soil enzyme activity measurement, soil samples from the Iowa State Century Farm were tested. About 8.6 MHz decrement in resonant frequency was observed after an hour for the soil sample. To create a control test, the soil sample was autoclaved at 120° C. and the same experiment was repeated. There was no significant shift in resonant frequency after the control soil addition (FIG. 9a). To simulate soil with increased enzyme activity, a sample was spiked with 30 μl of 200 mg/ml bacterial protease and the same experimental procedure was conducted which resulted in 16 MHz resonant frequency shift. The soil data was fit with the digestion model and resulted in a k_{cat}=0.119 g_{substrate}/(g_{enzyme}·s) at an assumed enzyme concentration of 19.2 mg/ml. Because the concentration is not known, it is better to use the density of the soil (1.498 g/cm³ in this experiment, which is comparable to the values found in the literature (0.1-1.5 g/cm³)) and transforming the activity to a soil mass basis; for this experiment we find a k_{cat} of 0.00152 g_{substrate}(gₛₒᵢₗ·s) (FIG. 9b). At this stage we approximated in field testing by 3D printing a case capable of isolating the resonator from the soil environment. It includes a tray for the resonator (FIG. 9ci) and accompanying cap (FIG. 9cii) that when connected isolates the resonator from the surrounding soil and water dielectric and provides a small channel for the analyte. To improve casting of the substrate, we also include a spacer (FIG. 9ciii) that can be inserted into the center well (FIG. 9civ) and allows for casting of the gel. This is then removed for testing (FIG. 9cV).

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed may be resorted to by those of ordinary skill in the art, and that such modifications and variations are considered to be within the scope of embodiments of the present disclosure.

### Additional Embodiments

The following exemplary embodiments are provided, the numbering of which is not to be constmed as designating levels of importance:

Embodiment 1 provides a resonator for detecting enzymatic activity, the resonator comprising:
a resonator comprising:
   an electronically conductive segment;
   a polymeric component coating at least a portion of the electronically conductive segment; and
   a substrate for one or more enzymes, the substrate disposed on the polymeric component.

Embodiment 2 provides the resonator of Embodiment 1, wherein the electronically conductive segment comprises a metal.

Embodiment 3 provides the resonator of Embodiment 2, wherein the metal comprises copper, silver, gold, aluminum, alloys thereof, or mixtures thereof.

Embodiment 4 provides the resonator of any one of Embodiments 1-3, wherein the electronically conductive segment is free from continuous segments.

Embodiment 5 provides the resonator of any of Embodiments 1-4, wherein the electronically conductive segment is free of discontinuous segments.

Embodiment 6 provides the resonator of Embodiment 5, wherein a profile of the conductive segment is an Archimedean spiral comprising one or more rings spaced relative to one another.

Embodiment 7 provides the resonator of Embodiment 6, wherein a pitch between the rings is constant across the spiral.

Embodiment 8 provides the resonator of Embodiment 6, wherein a pitch between the rings is variable across the spiral.

Embodiment 9 provides the resonator of Embodiment 6, wherein a pitch between a first portion of the adjacent rings is constant across the spiral and a pitch between a second portion of the adjacent rings is variable across the spiral.

Embodiment 10 provides the resonator of any one of Embodiments 7-9, wherein the pitch is in a range of from about 0.1 mm to about 10 mm.

Embodiment 11 provides the resonator of any one of Embodiments 7-10, wherein the pitch is in a range of from about 1 mm to about 3 mm.

Embodiment 12 provides the resonator of any one of Embodiments 1-11, wherein the largest dimension perpendicular to the longitudinal direction of the electronically conductive segment is in a range of from about 5 mm to about 100 mm.

Embodiment 13 provides the resonator of any one of Embodiments 1-12, wherein the largest dimension perpendicular to the longitudinal direction of the of the electronically conductive segment is in a range of from about 15 mm to about 60 mm.

Embodiment 14 provides the resonator of any one of Embodiments 1-13, wherein a thickness of the of the electronically conductive segment is in a range of from about 0.1 mm to about 5 mm.

Embodiment 15 provides the resonator of any one of Embodiments 1-14, wherein a thickness of the of the electronically conductive segment is in a range of from about 0.5 mm to about 1.5 mm.

Embodiment 16 provides the resonator of any one of Embodiments 1-15, wherein the polymeric component coats from about 10 percent surface area to about 70 percent surface area of the electronically conductive segment.

Embodiment 17 provides the resonator of any one of Embodiments 1-16, wherein the polymeric component coats from about 20 percent surface area to about 33 percent surface area of the electronically conductive segment.

Embodiment 18 provides the resonator of any one of Embodiments 1-17, wherein a height of the polymeric component is in a range of from about 10 μm to about 50 μm.

Embodiment 19 provides the resonator of any one of Embodiments 1-18, wherein a height of the polymeric component is in a range of from about 20 μm to about 30 μm.

Embodiment 20 provides the resonator of any one of Embodiments 1-19, wherein the polymeric component comprises a polyimide.

Embodiment 21 provides the resonator of Embodiment 20, wherein the polyimide comprises a repeating unit having the structure according to Formula I:

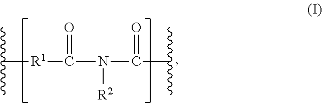

wherein
   R¹ is chosen from —O—, —NH—, and substituted or unsubstituted (C₁-C₂₀)hydrocarbylene; and
   R² is chosen from —H, —OH, and substituted or unsubstituted (C₁-C₂₀)hydrocarbyl.

Embodiment 22 provides the resonator of Embodiment 21, wherein the (C₁-C₂₀)hydrocarbylene is chosen from (C₁-C₂₀)alkylene, (C₁-C₂₀)alkenylene, (C₁-C₂₀)cycloalkylene, and (C₁-C₂₀)arylene.

Embodiment 23 provides the resonator of any one of Embodiments 21 or 22, wherein the (C₁-C₂₀)hydrocarbyl is chosen from (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, and (C₁-C₂₀)alkoxy.

Embodiment 24 provides the resonator of any one of Embodiments 1-23, wherein the substrate is in direct contact with the polymeric component.

Embodiment 25 provides the resonator of any one of Embodiments 1-24, wherein the substrate is disposed in a chamber.

Embodiment 26 provides the resonator of Embodiment 25, wherein the chamber comprises an O-ring contacting the polymeric component.

Embodiment 27 provides the resonator of any one of Embodiments 1-26, wherein the substrate comprises a cavity extending from a surface of the substrate to a surface of the polymeric component.

Embodiment 28 provides the resonator of any one of Embodiments 1-27, wherein the substrate is a substrate of a hydrolase enzyme.

Embodiment 29 provides the resonator of Embodiment 28, wherein the hydrolase enzyme is chosen from an esterase, nuclease, phosphodiesterase, lipase, phosphatase, DNA glycosylase, glycoside hydrolase, proteases, peptidase, acid anhydride hydrolase, helicase, GTPase, alcalase, and mixtures thereof.

Embodiment 30 provides the resonator of Embodiment 25, wherein the substrate comprises gelatin.

Embodiment 31 provides the resonator of Embodiment 25, wherein the substrate includes one component comprising a bond that is an ester bond, a glycosylic bond, an ether bond, a peptide bond, an acid anhydride bond, a halide bond, a phosphorous-sulfur bond, a sulfur-sulfur bond, a carbon-phosphorous bond, a carbon-sulphur bond, or a combination thereof.

Embodiment 32 provides the resonator of any one of Embodiments 28-31, further comprising the hydrolase enzyme.

Embodiment 33 provides the resonator of any one of Embodiments 1-32, further comprising a dielectric layer coating a portion of the electronically conductive segment that is free of contact with the polymeric component.

Embodiment 34 provides the resonator of Embodiment 33, wherein the dielectric layer comprises a dielectric material chosen from a bismaleimide-triazine (BT) resin, an epoxy resin, a polyurethane, a benzocyclobutene (BCB), a high-density polyethylene (HDPE), and combinations thereof.

Embodiment 35 provides the resonator of any one of Embodiments 33-34, wherein a major dimension of the dielectric layer is overlaps a major dimension of the polymeric component.

Embodiment 36 provides the resonator of any one of Embodiments 33-35, wherein a thickness of the dielectric layer is greater than a thickness of the polymeric component.

Embodiment 37 provides the resonator of Embodiment 36, wherein the thickness of the dielectric layer is in a range of from about 0.30 mm and about 2 mm.

Embodiment 38 provides the resonator of any one of Embodiments 36 or 37, wherein the thickness of the dielectric layer is in a range of from about 0.90 mm and about 1.1 mm.

Embodiment 39 provides the resonator of any one of Embodiments 1-38, further comprising a resonator reader for detecting a resonance frequency and a shift in resonance frequency of the resonator.

Embodiment 40 provides the resonator of Embodiment 39, wherein the resonator reader is positioned in line with the resonator.

Embodiment 41 provides the resonator of any one of Embodiments 39 or 40, further comprising a vector network analyzer connected to the reader.

Embodiment 42 provides the resonator of Embodiment 41, wherein the vector network analyzer is further connected to a computer.

Embodiment 43 provides the resonator of any one of Embodiments 1-42, further comprising an antenna coupled to the resonator.

Embodiment 44 provides the resonator of any one of Embodiments 1-43, wherein the resonator is a first resonator of a system and the system further comprises a second resonator.

Embodiment 45 provides the resonator of Embodiment 44, wherein a resonance frequency of the first resonator is different than a resonance frequency of the second resonator.

Embodiment 46 provides the resonator of any one of Embodiments 1 or 2, wherein the resonator comprises:
a continuous electronically conductive segment comprising copper;
a polymeric component comprising a polyimide at least partially coating the electronically conductive segment;
a substrate for the hydrolase enzyme disposed on the polymeric component; and
a dielectric layer comprising the epoxy resin contacting the electronically conductive segment that is free of the polymeric component.

Embodiment 47 provides a method of detecting enzymatic activity, the method comprising:
measuring a first resonance frequency of the resonator of the resonator according to any one of Embodiments 1-46;
contacting the substrate of the resonator with an enzyme; and
measuring a second resonance frequency of the resonator following contacting the substrate with the resonator.

Embodiment 48 provides the method of Embodiment 47, wherein the second resonance frequency is less than the first resonance frequency.

Embodiment 49 provides the method of any one of Embodiments 47 or 48, wherein at least one of the first resonance frequency and the second resonance frequency are in a range of from about 1 MHz to about 500 MHz.

Embodiment 50 provides the method of any one of Embodiments 47-49, wherein at least one of the first resonance frequency and the second resonance frequency are in a range of from about 1 MHz to about 100 MHz.

Embodiment 51 provides the method of any one of Embodiments 47-50, wherein the enzyme degrades the substrate.

Embodiment 52 provides the method of Embodiment 51, further comprising determining a rate of reaction between the enzyme and the substrate.

Embodiment 53 provides the method of Embodiment 52, wherein determining the rate of reaction between the enzyme and the substrate comprises measuring a plurality of resonance frequencies over a predetermined amount of time.

Embodiment 54 provides the method of any one of Embodiments 47-53, further comprising placing the resonator in at least one of soil, a tank, and a fabric.

Embodiment 55 provides a method of making the resonator according to any one of Embodiments 1-54, the method comprising:
providing or receiving the electronically conductive segment at least partially coated with the polymeric component;
etching the electronically conductive segment to form a pattern therein; and
contacting the polymeric component with the substrate.

Embodiment 56 provides the method of Embodiment 55, further comprising printing the pattern on a surface of the electronically conductive segment prior to etching.

Embodiment 57 provides the method of Embodiment 56, wherein the pattern is printed by an indelible marker.

Embodiment 58 provides the method of any one of Embodiments 55 or 57, wherein etching comprises at least partially immersing the electronically conductive segment in a solution comprising an etchant.

Embodiment 59 provides the method of Embodiment 58, wherein the etchant comprises hydrogen peroxide and hydrochloric acid.

Embodiment 60 provides the method of any one of Embodiments 55-59, further comprising forming a cavity in the substrate.

Embodiment 61 provides the method of any one of Embodiments 55-60, further comprising placing a mold on the polymeric component and forming the substrate therein.

What is claimed is:

1. A resonator for detecting enzymatic activity, the resonator comprising:
a resonator comprising:
an electronically conductive segment;
a polymeric component coating at least a portion of the electronically conductive segment; and
a substrate for one or more enzymes, the substrate disposed on the polymeric component.

2. The resonator of claim 1, wherein the electronically conductive segment comprises copper.

3. The resonator of claim 1, wherein a profile of the conductive segment is an Archimedean spiral comprising one or more rings spaced relative to one another.

4. The resonator of claim 1, wherein a pitch of the electronically conductive segment is in a range of from about 0.1 mm to about 10 mm.

5. The resonator of claim 1, wherein the largest dimension perpendicular to the longitudinal direction of the electronically conductive segment is in a range of from about 5 mm to about 100 mm.

6. The resonator of claim 1, wherein the polymeric component coats from about 10 percent surface area to about 70 percent surface area of the electronically conductive segment.

7. The resonator of claim 1, wherein the polymeric component comprises a polyimide.

8. The resonator of claim 7, wherein the polyimide comprises a repeating unit having the structure according to Formula I: 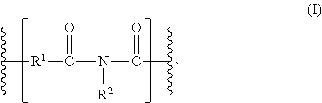
wherein
R¹ is chosen from —O—, —NH—, and substituted or unsubstituted (C₁-C₂₀)hydrocarbylene; and
R² is chosen from —H, —OH, and substituted or unsubstituted (C₁-C₂₀)hydrocarbyl.

9. A method of detecting enzymatic activity, the method comprising:
measuring a first resonance frequency of the resonator of the resonator of claim 1;
contacting the substrate of the resonator with an enzyme; and
measuring a second resonance frequency of the resonator following contacting the substrate with the resonator.

10. The method of claim 9, wherein the second resonance frequency is less than the first resonance frequency.

11. The method of claim 9, wherein at least one of the first resonance frequency and the second resonance frequency are in a range of from about 1 MHz to about 500 MHz.

12. The method of claim 9, wherein at least one of the first resonance frequency and the second resonance frequency are in a range of from about 1 MHz to about 100 MHz.

13. The method of claim 9, wherein the enzyme degrades the substrate.

14. The method of claim 13, further comprising determining a rate of reaction between the enzyme and the substrate.

15. The method of claim 14, wherein determining the rate of reaction between the enzyme and the substrate comprises measuring a plurality of resonance frequencies over a predetermined amount of time.

16. The method of claim 9, further comprising placing the resonator in at least one of soil, a tank, and a fabric.

17. A method of making the resonator of claim 1, the method comprising:
etching the electronically conductive segment at least partially coated with the polymeric component to form a pattern therein; and
contacting the polymeric component with the substrate.

18. The method of claim 17, further comprising printing the pattern on a surface of the electronically conductive segment prior to etching.

19. The method of claim 17, wherein etching comprises at least partially immersing the electronically conductive segment in a solution comprising an etchant.

20. The method of claim 17, further comprising forming a cavity in the substrate.
